(19) United States
(12) Patent Application Publication
(10) Pub. No.: US 2012/0330675 A1
(43) Pub. Date: Dec. 27, 2012

(54) REMOTE HEALTH CARE SYSTEM WITH STETHOSCOPE

(75) Inventor: Rajiv Muradia, Ottawa (CA)

(73) Assignee: Igeacare Systems, Inc., Richmond Hill (CA)

(21) Appl. No.: 13/418,580

(22) Filed: Mar. 13, 2012

Related U.S. Application Data
(63) Continuation of application No. 11/756,986, filed on Jun. 1, 2007, now abandoned.
(60) Provisional application No. 60/809,809, filed on Jun. 1, 2006.

(51) Int. Cl. G06Q 50/22; A61B 7/04

(52) U.S. Cl. 705/2; 381/67

(57) ABSTRACT

Disclosed is a patient health system for transmission of patient health data from a patient data collection system to a provider analysis system. The patient data collection system, located proximate a patient, is configured to collect patient physiological data and is operatively coupled to a communications network. The patient data collection system includes a patient data collection device couple to a patient work station. The patient work station is configured to transmit patient physiological data upon a determination that the communications network is reliable. The provider analysis system is coupled to the communications network and located remote from the patient data collection system.

### CROSS-REFERENCE TO RELATED APPLICATIONS

This application is a continuation of application Ser. No. 11/756,986, filed Jun. 1, 2007, which claims the benefit of provisional application Ser. No. 60/809,809, filed Jun. 1, 2006, which applications are incorporated herein by reference in their entirety.

### FIELD OF THE INVENTION

The invention relates to remote health care systems and more specifically, to remote monitoring of patient cardiac data captured with a cardiac sensor and transmitted over a communications network.

### BACKGROUND

Health care costs represent a significant portion of government budgets around the world. As the population ages and new expensive medical equipment and procedures are introduced health care costs continue to increase. Of particular concern, particularly in an older population, are cardiac problems because, unlike many other medical conditions, they often represent an immediate risk to the patient.

As is well understood in the art, a well-trained and experienced health care provider is often able to determine the existence of an undesirable condition by simply listening to a patient's body with a stethoscope. A stethoscope is used to detect and study heart, lung, stomach, and other sounds in adult humans, human fetuses, and animals. Using a stethoscope, the listener can hear normal and abnormal respiratory, cardiac, pleural, arterial, venous, uterine, fetal and intestinal sounds.

Stethoscopes vary in their design and material. Most are made of Y-shaped rubber tubing. This shape allows sounds to enter the device at one end, travel up the tubes and through to the ear pieces. Many stethoscopes have a two-sided sound-detecting device or head that listeners can reverse, depending on whether they need to hear high or low frequencies. Some newer models have only one pressure-sensitive head. The various types of instruments include: binaural stethoscopes, designed for use with both ears; single stethoscopes, designed for use with one ear; differential stethoscopes, which allow listeners to compare sounds at two different body sites; and electronic stethoscopes, which electronically amplify tones. Some stethoscopes are designed specifically for hearing sounds in the oesophagus or fetal heartbeats.

In the field of medical services, medical providers tend to be very busy and their time is very valuable. As a result, consultations with a medical providers such as cardiologists are often difficult to book and quite costly. In addition, due to the severity of some heart problems, it is not uncommon that suspected heart attack patients receive special treatment at emergency waiting rooms. This underscores the importance of early diagnosis for cardiac patients. In addition, it serves to demonstrate why treating such patients is often quite disruptive to medical facilities and, as a result, represents a significant cost to medical institutions.

It would be beneficial to provide a system that allows health care professionals to monitor a person who suspects that they have a heart problem absent booking an examination with a cardiologist.

### SUMMARY

Consistent with embodiments of the present invention, systems and methods are disclosed for transmitting patient physiological data representative of a patient's heartbeat captured by a patient health system located proximate a patient to a health care provider analysis system. The patient health system is comprised of a patient work station and a patient heartbeat sensor. The data representative of a patient's heart beat is collected by a patient heartbeat sensor located proximate a patient and operatively configured to collect patient physiological data and transmit the data collected to the patient work station. The patient work station is operatively coupled to a communications network that facilitates transmission of data representative of a patient's heart beat to the health care provider analysis system that is located remote from the patient health system. The communications network may be comprised of any data transmission medium such as, for example a broadband network, a wireless network, cellular network, satellite network or dial up network.

In one embodiment, the patient heartbeat sensor is comprised of an electronic stethoscope. The electronic stethoscope is coupled to a patient work station, which is operatively configured to determine the reliability of the communications network that facilitates transmission of patient physiological data representative of a patient's heart beat to the health care provider analysis system. The patient work station stores patient physiological data in temporary memory and upon a determination that the communications network is reliable gradually transmits patient physiological data in an orderly fashion.

It is to be understood that both the foregoing general description and the following detailed description are exemplary and explanatory only, and should not be considered restrictive of the scope of the invention, as described and claimed. Further, features and/or variations may be provided in addition to those set forth herein. For example, embodiments of the invention may be directed to various combinations and sub-combinations of the features described in the detailed description and include systems and methods for transmission of patient heart beat data from a patient work station to a remote health provider analysis system.

### BRIEF DESCRIPTION OF THE DRAWINGS

The invention is now described with reference to the drawings in which:

FIG. 1 is a diagram of an embodiment of the invention;

FIG. 2 is a illustration of an electronic stethoscope;

FIG. 3A. is an illustration of the process and flow of data that occurs during patient use of the system illustrated in FIG. 1;

FIG. 3B. is further illustration of the process and flow of data that occurs during patient use of the system illustrated in FIG. 1;

FIG. 4A. is an illustration of the process and flow of data that occurs during patient use of a remote stethoscope system as illustrated in FIG. 1;

FIG. 4B. is further illustration of the process and flow of data that occurs during patient use of a remote stethoscope system as illustrated in FIG. 1; and

FIG. 5. is an illustration of the process and flow of data that occurs during care provider use of the system illustrated in FIG. 1.

### DETAILED DESCRIPTION OF THE INVENTION

The following detailed description refers to the accompanying drawings. Wherever possible, the same reference numbers are used in the drawings and the following description to refer to the same or similar parts. While several exemplary embodiments and features of the invention are described herein, modifications, adaptations and other implementations are possible, without departing from the spirit and scope of the invention. For example, substitutions, additions or modifications may be made to the components illustrated in the drawings, and the exemplary methods described herein may be modified by substituting, reordering or adding steps to the disclosed methods. Accordingly, the following detailed description does not limit the invention. Instead, the proper scope of the invention is defined by the appended claims.

The present invention relates to systems and methods in the remote health care environment. Systems and methods consistent with embodiments of the present invention may be used to transmit data representative of a patient's heartbeat from a patient health system located proximate a patient to a health care provider analysis system located remote from the patient health system. The system and method may be facilitated by providing a sensor proximate the patient to sense the heart beat of a patient and generate data representative of a patient's heartbeat. In one embodiment, the sensor is an electronic stethoscope of the type that is currently available in the marketplace. The patient is required to position the stethoscope audio transceivers on their body in order to facilitate capture of the heartbeat sounds for storage on the patient work station and transmission to the work station. During use of the electronic stethoscope, the patient may be guided by the health care provider remotely via video transmissions to the patient work station which describes where the stethoscope audio transceivers are to be placed on the body. The patient may also be provided instructions on placement of the stethoscope audio transceivers by instruction positioned proximate the patient work station. The patient work station processes the data representative of a patient's heartbeat to determine if it is within predetermined acceptable ranges. Data representative of a patient's heartbeat that is not within acceptable ranges is flagged with an indicator so that a health care provider can easily pinpoint data which has been flagged. Next, the data representative of a patient's heartbeat along with any indicators is stored in a memory buffer. Upon a determination that the communications medium between the patient health system and the health care provider analysis system is sufficiently stable, the data within the memory buffer is transmitted in gradual and orderly fashion. Gradual and orderly data transmission helps to facilitate recovery and redistribution of incomplete data transmissions resulting from communication network service interruptions.

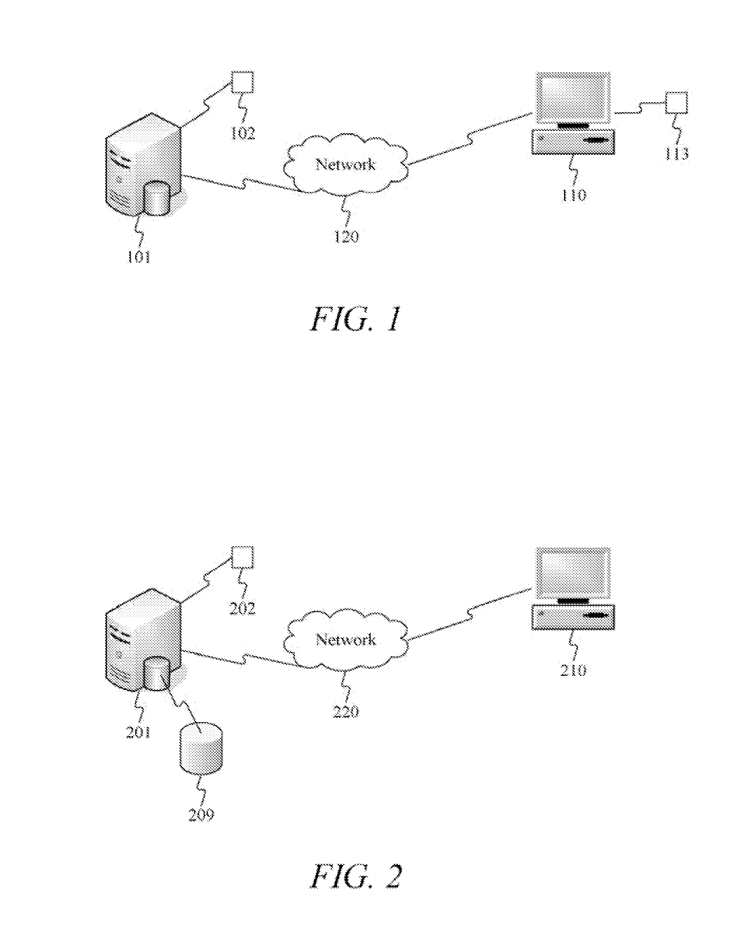

Consistent with an embodiment of the present invention, the aforementioned patient health system and health care provider analysis system operatively coupled via a communications network may be implemented in the embodiments illustrated in FIGS. 1 and 2. Referring to FIG. 1, a system according to the first embodiment of the invention is shown. The system 100 supports communication between a patient 101 and a server 110 of a health care provider that is remote to the terminal 101 and operatively connected via communications medium 120. The patient terminal 101 is operatively connected to a stethoscope 102 and an audio transceiver 103. The server 110 is operatively connected to an audio transceiver 113. A variety of different communications infrastructure is optionally used as the communications medium 120. For example, the terminal 101 optionally communicates with the server 110 via a wireless link, an Internet link or a plain old telephone system (POTS).

In use, the patient is provided instruction regarding locating the sensing portion of the stethoscope to provide good heartbeat information. Such instruction is optionally provided using a permanently printed card located proximate the terminal. Alternatively, the patient terminal 101 includes a display terminal configured to display a diagram of a human torso and an illustration of appropriate positioning of the sensing portions of the stethoscope. A communications path may be established between a medical professional proximate the server 110 and a patient proximate the patient terminal 101. The medical professional requests the patient hold a sensing portion of the stethoscope 102 proximate their heart such that their heart beat is monitored. The patient does so. Information regarding the heartbeat is then provided to the medical professional via the communications path and the server 110. The medical professional is then provided with the heartbeat information. If the test does not provide information sufficiently useful to determine if the patient has a problem the medical professional optionally requests that the patient run the test again. Alternatively, if the heartbeat is suggestive of a medical problem the patient is requested to take additional tests. Optionally, the patient terminal 101 is designed to support additional medical tests however this need not be the case. If further testing is desired the patient is optionally requested to visit the medical professional for the additional tests.

A person of skill in the art will appreciate that the system according to the first embodiment of the invention is useful when a patient undergoes circulation problems and their heartbeat is affected. Under such conditions it is clearly recommended that they seek appropriate attention. In this way, the first embodiment of the invention is a useful triage tool to assessing the severity of a heart condition before the patient is fully aware of the situation.

A person of skill in the art will appreciate that a stethoscope is a sensor that is useful in monitoring the heartbeat of a patient. Traditionally, a stethoscope operates as an audio transmitter allowing a medical professional, such as a doctor to hear the sound of a heartbeat without putting their ear against the chest of the patient as was once common practice. Clearly, a wide variety of configurations of sensors are optionally used to gather information regarding the quality of a heartbeat. Today, electronic stethoscopes are available with a variety of functionality an analysis tools. It will be clear to one of skill in the art that using an electronic stethoscope as the heartbeat sensor within the present invention allows for the functionality and analysis tools to be disposed at the patient terminal 101 or remotely at the health care provider location between the communication path 120. Alternatively, the tools are available from a third party. Optionally, tools provided by a third party are made available via the communications medium 120.

Thus, a doctor specializing in heart conditions is able to listen to a heartbeat and then choose one of a variety of heart data analysis tools for analyzing the heartbeat. Clearly, an experienced professional is properly suited to choosing the correct tools for assessing cardiac information. Optionally, the use of the tools is accompanied by a financial transaction. It is also contemplated that a heart beat analysis module may be installed on the patient terminal and configured so that patient heart beat data retrieved by the stethoscope may be processed such that, if certain heartbeat characteristics indicative of a danger to the patient are noted in the processing of the patient heartbeat data, the data is flagged. Further optionally, individuals, at least a medical professional are alerted when the data is flagged. Some of the individuals may be alerted simply to advise that there is a problem and others, such as medical professionals may be alerted so that the flagged data may be assessed.

A person of skill in the art will appreciate that a variety of different communications media are optionally used in accordance with the first embodiment of the invention. Thus, by providing additional ways for the medical professional to interact with the patient, the patient receives a higher quality remote health care experience. In addition, a remote terminal supporting a variety of communications media is optionally used to support other remote health care applications as is well understood in the art. In addition, FIG. 1 shows the stethoscope 102 tethered and thereby electronically coupled to the terminal 101. It is contemplated that the stethoscope 102 need not be tethered to the patient terminal 101 and may provide heartbeat information to the terminal via a wireless link.

Referring to FIG. 2, an alternative to the first embodiment of the invention comprises a memory buffer 209 disposed electronically proximate the patient terminal 201. The memory buffer 209 supports receiving data from the electronic stethoscope 202. The memory buffer 209 is optionally located within the terminal 201. In use, a patient uses the stethoscope 202 and records their heartbeat. The memory buffer 209 stores information received regarding the heartbeat. A person of skill in the art will appreciate that the ability to support communications between remote locations is often difficult to achieve in practice, particularly in areas that are not well served. The memory buffer 209 serves to mitigate such problems by storing information associated with the patient's heartbeat and providing it to the medical professional when the communications network supports such data transfer.

When patient heartbeat data is being transmitted between the terminal 201 and the server 210, the data is stored in the memory buffer 209 and transferred to the server 210 in a gradual fashion that supports verification of the accuracy of the provided information. In this way, should communication between the terminal 201 and the server 210 fail, the information regarding the heartbeat of the patient is still available. Optionally, the memory buffer 209 supports downloading of data stored therein via a local communications port, such as a universal serial bus (USB) port. A person of skill in the art will appreciate that buffering and then transmitting the heartbeat information will require more time than simply sending the heartbeat information directly. Clearly, in situations that allow the heartbeat information to be provided directly it is still beneficial to temporarily and simultaneously store the heartbeat information in the memory buffer 209 as even robust communications links are subject to temporary reductions in bandwidth and other types of failure. Optionally, the data transmitted is transmitted in a compressed form.

Further optionally, the patient terminal 301 comprises a computing device and a non-volatile storage medium. The non-volatile storage medium comprises predetermined medical instructions regarding how a patient holds a stethoscope to record their own heartbeat. When the patient accesses the terminal 301 they identify themselves. The computing device within the terminal interprets data within the non-volatile memory and provides the predetermined medical instructions to the patient in accordance with the data. The patient then acts to record their heartbeat in accordance with the instructions. Data within the memory buffer is later transmitted to the server 210. Optionally, the server 210 includes a memory that stores the transmitted data. This alternative embodiment is highly beneficial because it provides many of the benefits of the first embodiment of the invention absent the medical professional being immediately available. In this embodiment, a patient is able to record information that is useful in the diagnosis of heart ailments when they suspect that their may be a problem. Optionally, the terminal 201 comprises a feedback system that provides information to the patient regarding where to hold the stethoscope as well as other instructions, such as, when to hold their breath.

Optionally, the terminal 201 supports additional medical testing equipment, such as a heart rate monitor and blood glucose meter, to name a few. Such instruments are designed to support providing measured health information to the server 210. A person of skill in the art will appreciate that the first embodiment of the invention is easily modified to support a wide variety of tests.

Alternatively, the terminal 201 may include a video screen for providing visual information. In use, the medical professional is able to provide video information to the patient. Thus, should the patient experience some difficulty with a self-administered medical procedure, the medical professional is able to provide the patient relevant instruction both visually and audibly in order to assist the patient. Optionally, the medical professional provides a predetermined video stream to the terminal 201 where the media stream comprises medical instruction information for the purpose of instructing a patient regarding a self administered medical procedure. Further optionally, a set of such procedures are stored in a non-volatile storage memory proximate the server 210.

A person of skill in the art will appreciate that there are a wide variety of techniques for using a stethoscope. While one embodiment of the invention features a stethoscope that comprises a microphone that supports recording of heartbeat data to an external medium, an alternative stethoscope comprises an electronic microphone that is placed in close proximity to the patient's chest. In an alternative embodiment, the stethoscope comprises an elastic loop with a microphone that the patient positions against their skin proximate the heart with the elastic loop going around the chest. Such an embodiment optionally comprises a tension sensor for providing information regarding the amount of tension used to hold the sensor against the chest. The tension sensor facilitates the sensors ability to provide relatively consistent measurements. Further optionally, video or a picture of the patient wearing the stethoscope is taken such that if the stethoscope is poorly located the medical professional will be able to easily verify this and redirect the patient concerning proper positioning. Alternatively, the stethoscope comprises a shirt that the patient wears. The shirt has a plurality of sensors disposed proximate the expected location of the heart. In this way, one or more of the sensors is then used to obtain heartbeat information where the sensors are chosen based upon predetermined criteria. Optionally, the shirt comprises a mechanism for exerting some pressure against the skin of the patient to support proper contact between the skin and the sensors.

Optionally, an actuator, responsive to a signal provided remotely, is used to position the stethoscope. In this way, a medical professional is able to position the stethoscope in a fashion analogous to the way they would position the stethoscope manually in a face-to-face consultation. As is understood in the art it is beneficial to have the patient relax when heartbeat data is acquired and therefore, by positioning the stethoscope with an actuator, better heartbeat data is likely to be acquired.

It will be apparent to one of skill in the art that in many cases it is beneficial to have a medical professional other than cardiologist record stethoscope information and, should the medical professional suspect that there is a cardiac problem, provide the recorded heartbeat information to a cardiologist. In this way, the medical professional and the cardiologist have the opportunity to review the suspect heartbeat together, either in person or via a telecommunications conference. This has the additional benefit of teaching the medical professional the characteristics of a suspect heartbeat.

A person of skill in the art will appreciate that a wide variety of techniques are available to support communication between the terminal 201 and the server 210. Clearly, the choice of the technologies used is dependent upon a variety of factors, many of which are outside the scope of the present invention. Further, a person of skill in the art will appreciate that the embodiments of the invention presented are intended to be illustrative of the invention and not limiting. Numerous other embodiments of the invention will be apparent to one of skill in the art.

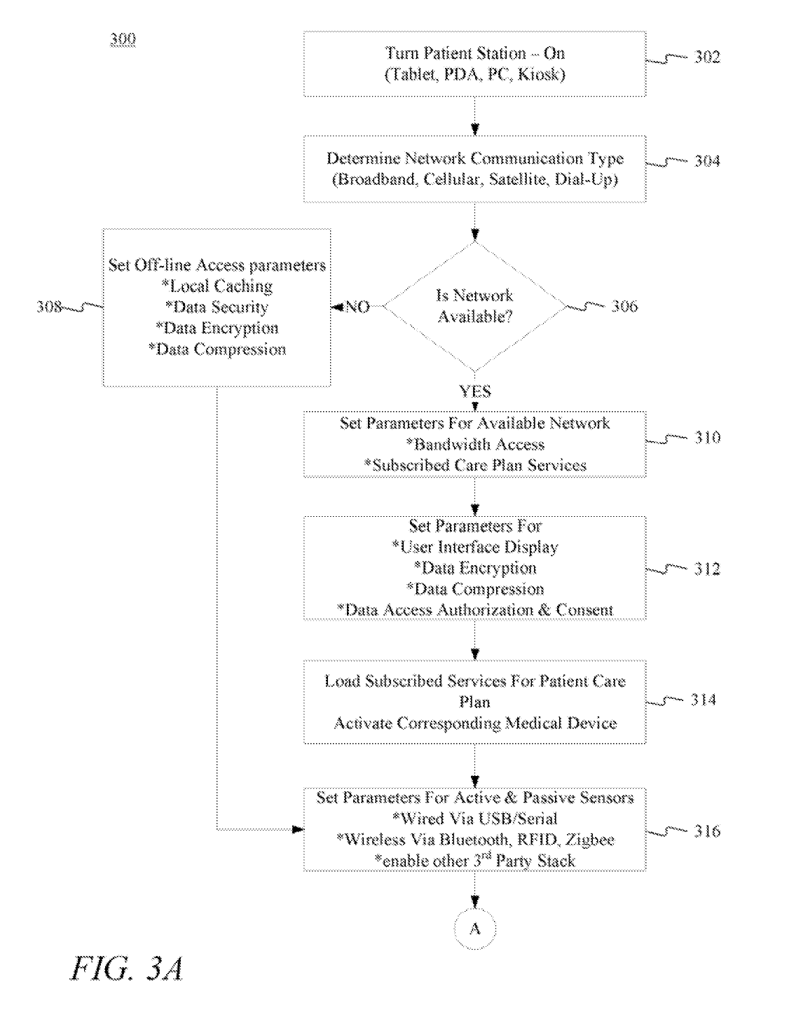

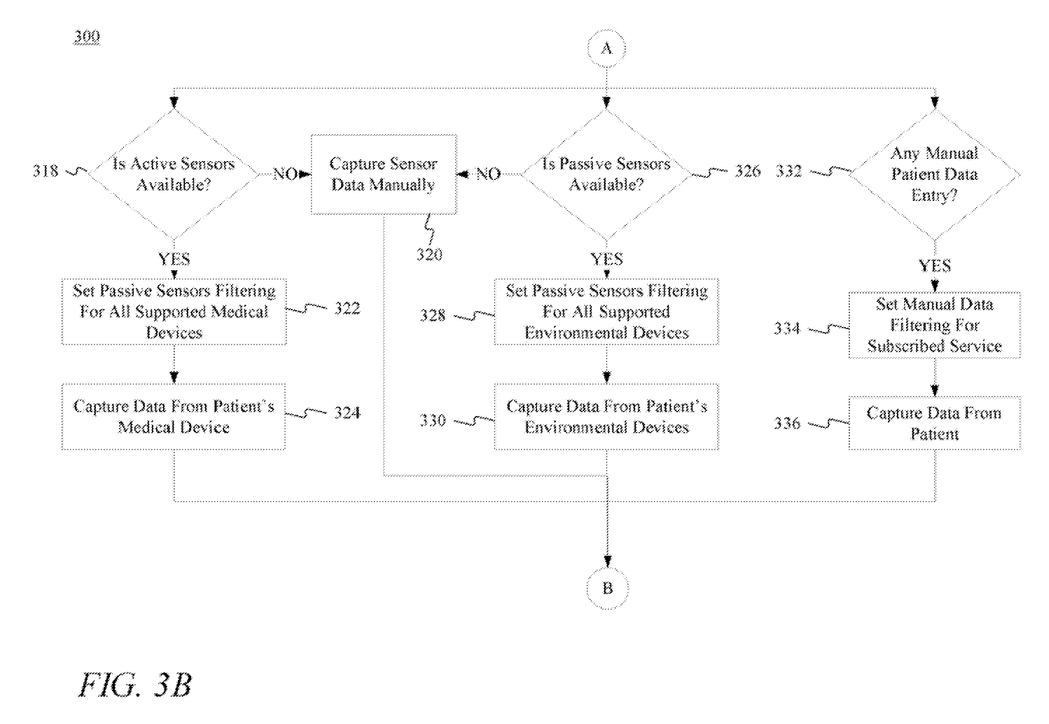

Referring now to FIG. 3A, the patient station which is a remote device utilized to enter patient physiological data remotely, may be any one of the following devices: a tablet PC, a PDA, a personal computer, a Kiosk laptop or any other computer-implemented configuration including a display screen, processor and memory. When operating a patient station, initially the device must be turned on 302. Upon activating the patient station, a communications link test is performed 304 to determine the network communication type across which the patient station shall transmit patient data. It is to be understood that the network communication type may be a wide area network that includes dialup (56 k), ISDN, T1, DSL, broadband, cellular, satellite, or any other communications medium that facilitates the transmission of data. The communications link test software module that checks the network communication type performs an assessment of which communication types may be available and also selects the optimal communications network if more than one communications network type is detected. For example, it is contemplated that there may be patient stations that include both dialup and broadband network communications. The communications link test software module that checks the network communication type selects the optimal network communication type and then determines whether the communications network selected is available 306. If the network is not available 308, the communications link test software module sets up the patient station to operate in offline mode.

During offline mode the patient may still use the workstation, even though there is no network communication between the patient station and the remote healthcare management server. However, the patient may interact with the patient station graphical user interface application to input data manually and to facilitate automatic capture of data from active and passive sensors. Data input during offline mode is locally cached with proper data security. Alternatively, if the communications network is available 306, the patient station sets parameters for transmitting data across the available communications network. Next, the patient station determines the type of care plan services the patient has access to 310. The care plan services may include services such as video visit, vital signs monitoring, blood pressure monitoring, blood glucose monitoring, blood oxygen monitoring, body weight monitoring, body temperature monitoring, pulmonary function analysis, respiratory monitoring, neurological monitoring, cardiac monitoring, sleep monitoring bathroom visit monitoring, bedroom visit monitoring, activity monitoring (sensors in the house), meal preparation monitoring air quality monitoring, patient fall status monitoring (sensors to detect body up/down position) or any other services that may be available to a patient via the patient workstation. It is to be understood that the care plan services that are active as icons on the patient station shall be configured by the care provider remotely or upon the patient station prior to delivery. The patient workstation is configured for the patient based on the patient's illnesses and the services that a patient may require. For example, if a patient is diabetic, the patient station shall be configured to interface with a glucose meter and a weight scale and have the medication reminder service. By way of further example, if the patient is a cardiac heart failure patient (CHF), the patient station shall be configured to interface with a stethoscope as well as an apparatus for capturing the patient's ECG measurements.

Following a determination by the patient workstation that the network is available, a determination is made by the patient station configuration module of the bandwidth for the communications network and the services which may be pushed on that bandwidth. Next the system sets the patient workstation for user interface display 312. If the network communication type is dialup, a patient would not be able to facilitate wound management interface, because wound management includes a video component. If the network communication is high-speed DSL, wound management is an application which may be engaged because the video component may be streamed via the high-speed DSL connection. For example a patient having diabetes, may subscribe to the wound management service and thereby have a wound management icon display on the patient station. The wound management service allows wounds to be displayed and recorded by the healthcare provider. Typically during operation, a patient station camera is utilized to facilitate capture of ulcers on the feet of the patient for transmission back to the server of the healthcare provider system. The images are transmitted from the patient station back to the server of healthcare provider system at which a nurse may be stationed for viewing the images to provide feedback which may be immediate when images are viewed as they are being streamed across the communications network or at a later time when the video images are stored in server memory.

Next, the patient station configuration module sets the parameters for user interface display, data encryption, data compression, and data access authorization and consent 312. The data encryption parameters being utilized is a key pair encryption. A key that is stored on the healthcare provider's server is utilized to encrypt the data. Utilization of a key pair encryption guarantees that data transmitted over the communication network cannot be intercepted and viewed by individuals intercepting data being transmitted over the communications network. Data compression is performed to facilitate shrinking of data so that the data can be transmitted on a network having very low bandwidth. For example if the network is a dialup network the data can be compressed and transmitted at a faster rate. The compression algorithm is a standard application protocol interface (API). Data access authorization and consent is the control mechanism whereby the system dictates the individuals who have access to and can actually look at the patient data once it is captured. The data access authorization and consent parameters define the individuals whom may have access to patient data. Data access authorization and consent parameters are defined on the patient station. For example a patient may define the parameters such that his or her pharmacist does not have access to the patient's physiological data representative of the patient's vital signs. However, the pharmacist may have access to data concerning a patient's diet, medication plan and any other data which the patient determines that the pharmacist needs to have access.

Next, services to which the patient subscribes are loaded on onto the patient station by loading the icons that correspond to a subscribed service onto the patient station 314. Based on the icons loaded onto the patient station, active and passive sensors that correspond to the service icons loaded may be activated by engaging the icons. For example, an icon that is loaded onto the patient workstation is to facilitate glucose monitoring. That icon has to be operatively connected to a sensor, which in this example is an active sensor, such as a glucose monitor. For glucose monitoring interface to be fully functional on the patient station, the glucose monitor must be activated and operatively connected to the patient workstation. In one embodiment operative connection and activation may be performed by Bluetooth communications. Next, parameters are set for active and passive sensors 316. Engaging the subscriber service icon causes the parameters for the active and passive sensors to be set 316. It is contemplated that active and passive sensors may be connected or communicating with the patient workstation via wired USB or serial connections, wireless Bluetooth, RFID or Zigbee communications or any other third party communications protocol. The Bluetooth communications link is performed by pairing the workstation with the active or passive sensor in accordance with normal Bluetooth pairing protocol.

Following the setup of the parameters for active and passive sensors, in accordance with the services associated with a patient, the system tries to determine whether any active or passive sensors are available 318, 326. In the case of a diabetic patient they have engaged the icon for measuring their blood sugar level through use of the glucose monitor, an active sensor. Upon a determination that there are active sensors 318, a filtering mechanism 322 is engaged to make sure that only the proper data is being pulled into the patient workstation. Proper data is data that falls within previously defined minimum and maximum range levels. Data falling within the acceptable range is captured and stored on the patient station. When data received is above or below the range of acceptable data, the data is flagged and saved. An alert is also attached to the data and the alert is transmitted to the remote healthcare provider system to indicate a potential patient health issue or a problem with the active sensor.

Upon a determination that there are passive sensors 326, a filtering mechanism 328 is engaged to make sure that only the proper data is being pulled into the patient workstation. Proper data is data that falls within previously defined minimum and maximum range levels. Data falling within the acceptable range is captured and stored on the patient station. When data received is above or below the range of acceptable data, the data is flagged and saved. An alert is also attached to the data and the alert is transmitted to the remote healthcare provider system to indicate a potential patient health issue or a problem with the passive sensor. Passive sensor may be devices operatively connected to the patient station to determine for example, the opening and closing of a washroom door, or the CO₂ levels in the home.

The system is also capable of facilitating manual data entry 332. For example if a patient needs to enter their temperature into the patient station, because thermometers are not Bluetooth capable nor do they have USB or any other communications capability, the user must enter data representative of the patient's temperature into the patient station manually. The patient station includes a keypad whereby the patient may enter the value that the patient sees on the medical device. Following a determination that there is data for manual data entry 332, a filtering mechanism 334 is engaged to make sure that only proper data is being pulled into the patient workstation. Proper data is data that falls within previously defined minimum and maximum range levels. Data falling within the acceptable range is captured and stored on the patient station. When data received is above or below the range of acceptable data, the data is flagged and saved. An alert is also attached to the data and the alert is transmitted to the remote healthcare provider system to indicate a potential patient health issue or a problem with the device for which data has been entered.

The patient data captured by the patient station is stored in a local cache for the store forward transmission function 338. Next, the data for each service is displayed in a visualizer to facilitate graphic representation of captured patient data 340. Next the system checks to determine if the communications network is online or available 342. If the network is available the patient workstation synchronizes and transmits patient data with the central server 344.

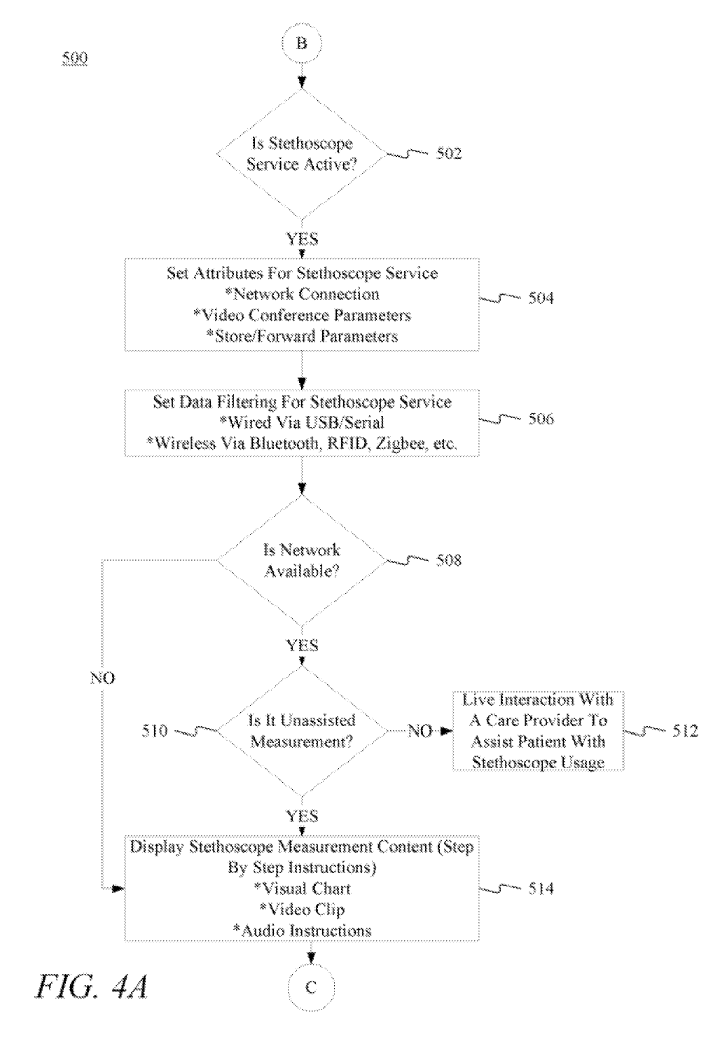

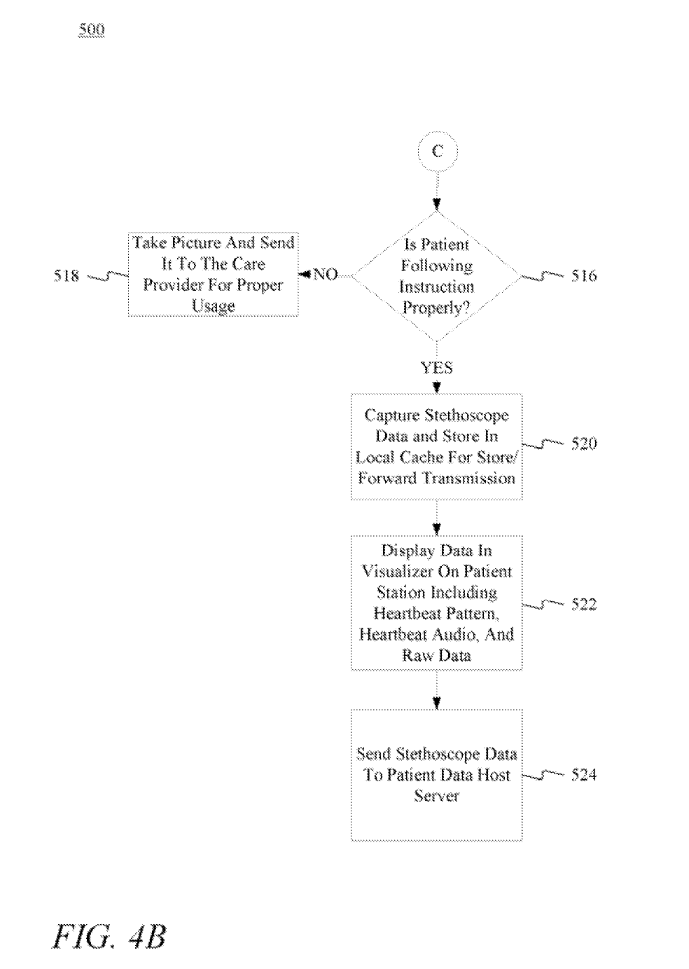

The central server 344 serves as a centralized data repository to which health care providers and other individuals who have been granted authorization and consent by the patient to certain data files may connect and gain access to information to which they have authorization. As illustrated in FIG. 4, health care providers may connect to the central server 402. Connection may occur via WAN, but is generally done via a web based Internet connection. This application is simply a web browser that individuals enter and gain access to in response to the entry of their respective credentials. Upon gaining access to the web browser, the user receives displays, alerts and messages based on their respective authorization and consent previously defined by the patient 404. The web browser facilitates access to the centralized data repository by allowing users to login and gain access to files based on the authorization and consent provider a user by the patient 406. The health care provider seeking access to the central server may be a network of care providers including any of the following individuals, nurse, primary physician, pharmacist, family members, etc. These individuals each have access to certain subsets of the patient data based on the authority assigned at the access authorization and consent previously defined 406.

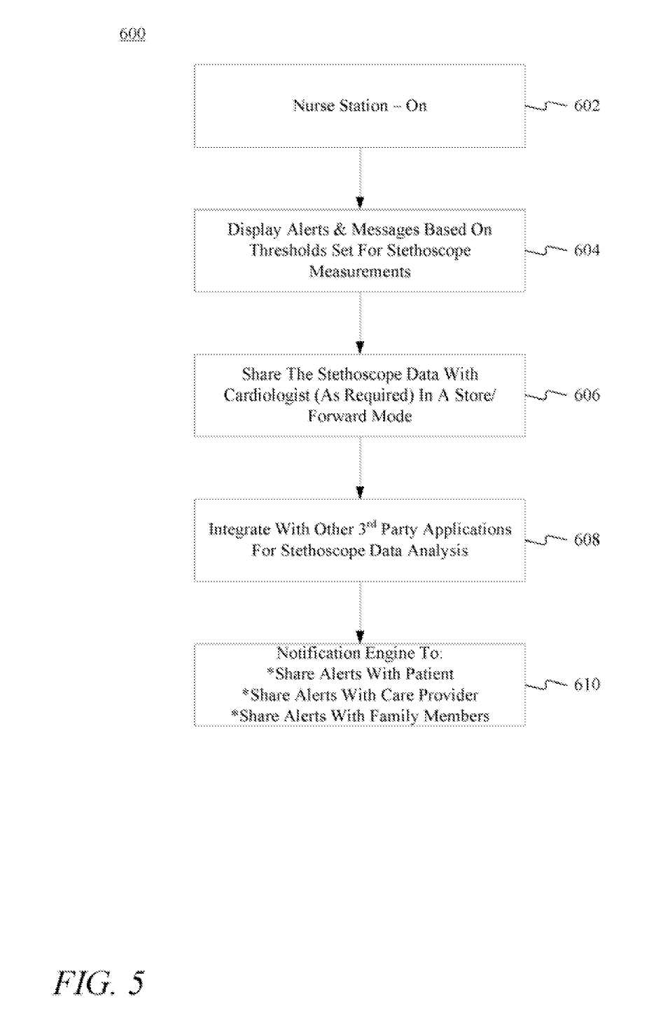

FIG. 5 illustrates patient station data processing when the active filter is a stethoscope. The stethoscope that is used in the present invention may generally be referred to as an electronic stethoscope. This stethoscope is not the binaural stethoscope designed for use with both ears that one visualizes when they hear the word stethoscope. It is configured with head that listeners that are connected to a jack that plugs into an audio port on the patient station. As FIG. 5 illustrates, if the active sensor is a stethoscope, to which the patient has subscribed service 502, a determination is made as to whether the patient subscribes to stethoscope service by engaging the stethoscope icon on the patient station GUI. If the patient subscribes to stethoscope service, the patient station operations module sets the attributes for stethoscope service 504 by checking the network connection, matching the attributes of stethoscope service with the network connection and setting the attributes for the store forward function. Regarding matching the attributes of stethoscope service with the network connection, if the network connection is Broadband for example, the patient station operations module sets attributes to allow video conferencing. If the network connection is dial up, the patient station will not set the attributes to allow video conferencing because the network connection does not support such service. Regarding the setting of attributes for the store forward function, these attributes define how much of the audio stream needs to be stored in order to facilitate safe data transmission which allows for recovery of data during faulty transmission or service interruption. For example, the amount of data that needs to be stored in the local cache before being forwarded depends on whether data is to be transmitted across a broadband connection network or a dial up connection.

In one embodiment, when the communications network is dial up, data is stored in 10 second groupings and forwarded. When the communications network is broadband, data packets are stored in 30 second blocks and forwarded. The 30 second block of data packets are transmitted across the communications network in an orderly fashion. Patient station data processing includes an algorithm that tracks the data packets being sent and includes a verification mechanism for verifying that all data packets transmitted within a 30 second block were received. The verification mechanism is the transmission of an acknowledgement that is sent back to the patient station from the central server following verification by the algorithm that the entire 30 second block of data packets was received. The algorithm determines whether a block of data packets has been received by the size of the block of data packets. For example a first 30 second block of data packets is created then sent, a second 30 second block of data packets is created then sent, a third 30 second block of data packets is created then sent and so on. This helps facilitate maintaining the integrity of the data so that if there is a connection loss during transmission of the second 30 second block of data packets, no other data shall be transmitted until the connection has been reestablished. Upon reestablishing the connection, the entire second 30 second block of data packets shall be sent again and a third 30 second block of data packets will then be sent behind the second 30 second block of data packets in the previously defined sequence.

Next, the patient station enables a filtering mechanism 506 for the stethoscope in order to make sure that only the proper data being transmitted from the electronic stethoscope into the patient workstation is being stored. Proper data is data that falls within previously defined minimum and maximum range levels. Data falling within the acceptable range is captured and stored on the patient station. When data received is above or below the range of acceptable data, the data is flagged and saved. An alert is also attached to the data and the alert is transmitted to the remote healthcare provider system to indicate a potential patient health issue or a problem with the electronic stethoscope. In addition, the patient work station determines whether the electronic stethoscope is communicates by wire or wirelessly. Communication that occurs via wire, a determination is made as to whether the wire connection is USB or serial connection. For communication that occurs wirelessly, a determination is made as to whether the communication is Bluetooth, RFID, Zigbee or some other third party wireless protocol. Next, a determination is made as to whether a communications network is available 508. If the communications network is available, the patient station determines whether the patient data to be collected will be unassisted (without health care provider support) 510.

If the patient data collection by the stethoscope is to be assisted, there will be live interaction with a care provider to assist the patient with stethoscope usage 512. If the patient data collection by the stethoscope is to be unassisted, the patient station displays stethoscope measurement content 514, providing step by step instructions on positioning of the stethoscope on the body. These instructions may be provided as a visual chart, video clip or audio instructions 514. Next, the patient station determines whether the patient is following the instructions 516. If the patient is not following instructions properly, the system takes a picture and sends it over to the care provider for use in helping to assist the patient in advising on the proper way to position the stethoscope on the body. If the patient is following instructions properly, the patient station captures the stethoscope data and stores it in local cache for the store and forward patient data capture process 520. Next, the stethoscope data is displayed on the patient station through use of a visualizer application that facilitates graphic representation of captured patient data 522. The visualizer displays the patient heart beat data as a visual pattern, audio file and as raw heartbeat data. Next the system checks to determine if the communications network is online or available. If the network is available the patient workstation synchronizes and transmits patient data with the central server which may be located at a host location or at the location of the health care provider 406.

FIG. 6 illustrates the system operation at a nurse station remote from the patient station. The nurse's station which is turned on 602 shall display alerts and messages based on the thresholds set for stethoscope measurements 604. All data that has come in, if it is valid, it is stored locally within the central server within the patient records. This facilitates the nurse being able to share the information with a health care specialist such as a cardiologist 606. Next, the raw data recorded by the stethoscope may be integrated with and processed by a third party application that performs stethoscope data analysis 608. For example an application that may determine whether the patient is having some form of arrhythmia based on the stethoscope data. Next the central server takes all the alerts and messages transmitted from the patient station and send it through a notification engine that allows the alerts to be shared with a predefined class of individuals such as the patient, care providers or family members.

While certain features and embodiments of the invention have been described, other embodiments of the invention will be apparent to those skilled in the art from consideration of the specification and practice of the embodiments of the invention disclosed herein. Furthermore, although embodiments of the present invention have been described as being associated with data stored in memory and other storage mediums, one skilled in the art will appreciate that these aspects can also be stored on or read from other types of computer-readable media, such as secondary storage devices, like hard disks, floppy disks, or a CD-ROM, a carrier wave from the Internet, or other forms of RAM or ROM. Further, the steps of the disclosed methods may be modified in any manner, including by reordering steps and/or inserting or deleting steps, without departing from the principles of the invention.

It is intended, therefore, that the specification and examples be considered as exemplary only, with a true scope and spirit of the invention being indicated by the following claims and their full scope of equivalents.

What is claimed is:

1. A patient health system for transmission of patient health data from a patient data collection system to a provider analysis system, comprising:
a. the patient data collection system, located proximate a patient, configured to collect patient physiological data, and operatively coupled to a communications network;
b. the provider analysis system coupled to the communications network and located remote from the patient data collection system;
wherein the patient data collection system is comprised of a patient data collection device couple to a patient work station, the patient work station being configured to determine the reliability of the communications network and transmit patient physiological data upon a determination that the communications network is reliable.

2. A patient health system for transmission of patient heart beat data from a patient data collection system to a health provider analysis system, comprising:
a. the patient heartbeat data collection system, located proximate a patient, configured to collect patient physiological data representative of patient heart beat, and operatively coupled to a communications network;
b. the health care provider analysis system coupled to the communications network and located remote from the patient heart beat data collection system;
wherein the patient heart beat data collection system is comprised of an electronic stethoscope coupled to a patient work station, wherein the patient work station is configured to determine the reliability of the communications network and transmit patient physiological data upon a determination that the communications network is reliable.

3. The patient workstation of claim 2 comprising a data input device, data display device, processor, memory and an audio transceiver.

4. The patient workstation of claim 2 whereby the memory includes a temporary memory buffer.

5. The patient workstation of claim 2 configured to provide the patient with instructions regarding positioning of stethoscope audio receiver on the patient.

6. A method of transmitting data representative of a patient's heartbeat from a patient data collection system located proximate a patient to a health care provider analysis system comprising:
a. providing a sensor proximate the patient to sense the heart beat of a patient and generate the data representative of a patient's heartbeat;
b. processing the data representative of a patient's heartbeat to determine if it is within an acceptable range;
c. flagging portions of the data representative of a patient's heartbeat that is outside of the acceptable range;
d. storing the data representative of a patient's heartbeat along with any flagged portions in a memory buffer;
e. determining if a communications medium between the patient data collections system and the health care provider analysis system is sufficiently stable for data transmission;
f. transmitting the data representative of a patient's heartbeat along with any flagged portions to the health care provider analysis system upon a determination that the communications medium is stable.

7. The method of claim 6 wherein the sensor is a stethoscope.

8. The method of claim 6 wherein a first audio transceiver is further provided proximate the patient to facilitate audio communications between the patient and the health care provider analysis system that includes an audio transceiver.

9. The method of claim 6 wherein the data transmitted to the healthcare provider analysis system over a communications medium is digital data.

10. The method of claim 6 wherein the communications medium comprises a wireless network.

11. The method of claim 6 wherein the communications medium comprises a broadband network.
